(19) United States
(12) Patent Application Publication
(10) Pub. No.: US 2020/0405505 A1
(43) Pub. Date: Dec. 31, 2020

(54) Joint Replacement And In Situ Gauge System

(71) Applicant: DUGGAN INNOVATIONS LLC, Corville, AZ (US)

(72) Inventors: Brian Thomas DUGGAN, Cornville, AZ (US); Kevin Jason NACKARD, Flagstaff, AZ (US)

(73) Assignee: Duggan Innovations LLC, Cornville, AZ (US)

(21) Appl. No.: 16/490,195

(22) PCT Filed: Apr. 12, 2019
(86) PCT No.: PCT/US2019/027348

§ 371 Date: Aug. 30, 2019

Related U.S. Application Data
(60) Provisional application No. 62/664,253, filed on Apr. 29, 2018.

(51) Int. Cl. A61F 2/46

(52) U.S. Cl. CPC A61F 2/4657; A61F 2/461; A61F 2002/30131; A61F 2002/4658; A61F 2002/4661; A61F 2/4612

(57) ABSTRACT

A joint replacement in situ gauge system enables a surgeon to measure the appropriate spacing between the two sides of a joint, a convex component and a concave component. A replacement in situ gauge system includes a gauge component to measure the pressure and/or displacement of one joint component with respect to the mating component. A gauge component utilizes a displacement device that displaces the convex or concave component to determine a proper spacing for an implant component. The displacement component may be a bladder that is inflated with a fluid and a gauge may measure the pressure of the bladder through a range of motion. A scale may be used to measure physical displacement. The gauge component may be configured in the convex and/or concave components of the system. The gauge component may be removed and then an implant component may be implanted.

### CROSS REFERENCE TO RELATED APPLICATIONS

This application claims the benefit of priority to U.S. provisional patent application No. 62/664,253, filed on Apr. 29, 2018; the entirety of which is hereby incorporated by reference herein.

### BACKGROUND OF THE INVENTION

### Field of the Invention

The invention relates to a joint replacement in situ gauge system and a method of using the gauge system in a joint replacement procedure.

### Background

Joint replacement surgery frequently requires the implant of a ball component into a socket, or more generally, a convex component into a concave component. When replacing a convex and/or a concave component of a joint, proper alignment and displacement of these components with respect to each other is critical to enable a full range of motion. In an exemplary embodiment, a convex component having a convex, or curved surface such as a radiused surface including a spherical surface for example, is attached to a first bone. A concave component having a concave portion, or recessed curved surface or radiused surface, is attached to a second bone. The concave portion of the concave component is configured to nest around the convex component to reconstruct the joint. The socket, or concave portion, of the concave component and convex component must be configured with a proper amount of space or mating pressure to enable proper movement and range of motion of the joint and to prevent wear and pain. To achieve the proper spacing, a surgeon will install a concave component and, in some cases, an initial spacer for the concave component, and then test the range of motion of the joint. If there is too much pressure, the range of motion may be limited or it may be difficult to move the limb through a range of motion. If there is too much space between the convex component and concave component, the joint may be too loose and the convex component may slip out of position with respect to the concave component when the range of motion is evaluated. The surgeon will then try a different concave and/or convex component or a different sized spacer to modify the space between the convex and concave component and again, check the range of motion. This iterative method is somewhat subjective and subjects the joint to excess trauma. In addition, the iterative spacing method takes additional operating time and makes recovery more difficult.

### SUMMARY OF THE INVENTION

The invention is directed to a joint replacement in situ gauge system that enables a surgeon to apply a known amount of distraction force in order to measure the appropriate spacing between the two sides of a joint, a convex component and a concave component, for example. An exemplary joint replacement in situ gauge system comprises a gauge component which may be configured in a convex component or concave component. A gauge component comprises a displacement device that displaces the mating surfaces of the joint replacement in situ gauge system with a known amount of force to determine a proper spacing. The displacement component may be a bladder that is inflated with a fluid and a gauge may measure the pressure of the bladder. A specific or specified or range of pressures may be desired for a given patient and joint being replaced to achieve a desired distraction force. In addition, a surgeon may move the joint through a range of motion with the bladder inflated to evaluate the range of motion. A scale may be configured on the gauge component to indicate the proper size of the implant component, concave or convex. The scale may indicate an amount of displacement of the gauge component and this displacement may be used to select an appropriately sized implant component. A surgeon may then remove the gauge component or assembly and implant the properly sized permanent device. This process will reduce the need for iterative trials of implant components and reduce operating time and trauma to the patient.

In an exemplary embodiment, a joint replacement in situ gauge system comprises a gauge concave assembly that comprises a concave component having a concave portion that nests around an implant convex component. A reverse shoulder replacement joint typically has this configuration. A surgeon may implant the convex component on one bone and then configure the gauge concave assembly in the mating bone. A gauge concave assembly may fit into an implant stem insert and mimic the size and dimensions of the implant concave component. A displacement device may be configured between a plate portion and the gauge concave component to displace the gauge concave component toward the implanted convex component while remaining substantially parallel to the plate. A prescribed pressure or range of pressures may be determined for a given patient and joint to deliver a desired amount of distraction force. The surgeon may displace the gauge concave component to the prescribed pressure and measure the displacement. An exemplary gauge concave assembly may comprise a scale to indicate the displacement and the surgeon may visually determine the size of the implant concave component by viewing the scale at the prescribed pressure. The gauge concave component and/or assembly may then be removed and the permanent implant concave component having the required size may then be implanted and secured.

In an exemplary embodiment, a joint replacement in situ gauge system comprises a gauge convex component or assembly that comprises a convex component configured to nest into a concave portion of an implant concave component. A shoulder replacement joint typically has this configuration. A surgeon may implant the concave component on one bone and then configure the gauge convex assembly in the mating bone. A gauge convex assembly may fit into an implant stem insert and mimic the size and dimensions of the implant convex component. A displacement device may be configured between a plate portion and the gauge convex component to displace the gauge convex component toward the implanted concave component while remaining substantially parallel to the plate. A prescribed pressure or range of pressures may be determined for a given patient and joint to deliver a desired amount of distraction force. The surgeon may displace the convex component to the prescribed pressure and measure the displacement. An exemplary gauge convex assembly may comprise a scale to indicate the displacement and the surgeon may visually determine the size of the implant convex component by viewing the scale at the prescribed pressure. The gauge convex component and/or assembly may then be removed and the permanent implant convex component having the required size may then be implanted and secured.

In an exemplary embodiment, a replacement joint may comprise mating surfaces of varying concavities and convexities. A replacement knee joint typically has this configuration. A joint replacement in situ gauge system for a knee joint may comprise two separate displacement devices to enable individual determination of spacing between each of the convex components and concave components. With the pressure or force set to a desired level for each of the displacement devices, the spacing or displacement may be non-uniform. This non-uniform displacement may be required for proper load across the joint and to reduce wear and pain. It may be desirable to have uniform pressure or loads across the joint and therefore the size of the implant from the medial to the lateral side of the joint may be different.

An exemplary displacement device may comprise a bladder and a fluid such as a non-compressible liquid, such as saline, to inflate the bladder. Any suitable fluid may be used and preferably the fluid is sterile. A fluid may be a gas, such as air or nitrogen for example. Pressure can be increased in the bladder via mechanical device such as a balloon indeflator, and a force gauge may be used to measure the pressure of the fluid in the bladder. Because the pressure is applied to known geometry, a known relationship between pressure and distraction force exists A displacement device may be a mechanical displacement device and a strain gauge or other force measurement device may be configured to measure the force. A bladder type displacement device may be preferred to an electro-mechanical device as it may be disposable, or a single-use type of device. Exemplary bladder geometries may be spherical, cylindrical, or toroidal.

An exemplary gauge convex or concave assembly may comprise a displacement gauge for determining the amount of displacement of the convex or concave component from the plate portion. A displacement gauge may be a scale that is configured on the gauge assembly and the surgeon may view the amount of displacement on the scale to determine a proper sized implant device. A displacement gauge may comprise a sensor, such as a proximity sensor or laser displacement sensor that measure the amount of displacement of the convex or concave component from the plate portion and provides a read-out. An exemplary sensor may send the displacement value wirelessly to a receiver, such as an electronic device for readout, for example.

An exemplary gauge convex assembly may mimic the actual implant convex component or assembly and have substantially the same size and shape as the implant convex assembly except for the gauge convex component being able to be displaced toward the concave component. An exemplary gauge convex assembly may fit into a convex component stem so that it is retained in substantially the same position as the implant convex component, thereby enabling a direct measurement of displacement in situ. Likewise, an exemplary gauge concave assembly may mimic the actual implant concave assembly or component and have substantially the same size and shape except for the gauge concave component being able to be displaced toward the convex component. An exemplary gauge concave assembly may fit into a concave component stem so that it is retained in substantially the same position as the implant concave component, thereby enabling a direct measurement of displacement in situ. An exemplary gauge convex or concave component may comprise a plate component that sits on the face of the bone and the stem or stem insert may extend from the plate component. The displacement device may be configured between the plate component and the convex or concave component.

The exemplary gauge convex and concave component may be made out of implantable materials, and may be sterilized. Exemplary gauge convex and/or concave components may comprise stainless steel, titanium, fluoropoymer, polyethylene, other suitable polymers and elastomers including, but not limited to fluoro-elastomer materials such as synthetic rubber and fluoropoymer elastomer compositions, and the like.

While specific concave and convex joints have been disclosed herein, it is to be understood that the joint replacement in situ gauge system may be used in any suitable joint including the knee and shoulder joints as disclosed as well as elbow shoulder, hip, and may also be used between vertebrae.

The summary of the invention is provided as a general introduction to some of the embodiments of the invention and is not intended to be limiting. Additional example embodiments including variations and alternative configurations of the invention are provided herein.

### BRIEF DESCRIPTION OF SEVERAL VIEWS OF THE DRAWINGS

The accompanying drawings are included to provide a further understanding of the invention and are incorporated in and constitute a part of this specification, illustrate embodiments of the invention, and together with the description serve to explain the principles of the invention.

FIG. 1 shows a joint replacement system configured for a reverse shoulder joint having a convex component in the scapula and a concave component as the gauge component of the system in the humerus.

FIG. 2 shows an exemplary convex component assembly of the joint replacement system secured into the scapula by a stem.

FIGS. 3 and 4 show an exemplary gauge concave assembly of the joint replacement system secured into the concave receiver bone by a stem.

FIG. 5 shows an exemplary gauge concave component and stem assembly.

FIG. 6 show an exemplary gauge concave assembly being inserted into a stem secured in a bone.

FIGS. 7 and 8 show an exemplary gauge concave assembly having a displacement device to displace or move the concave component with respect to the plate portion. FIG. 8 shows the displacement device extending the concave component away from the plate portion.

FIGS. 9 and 10 show an exemplary gauge convex assembly having a displacement device to displace or move the convex component with respect to the plate portion. FIG. 10 shows the displacement device extending the convex component away from the plate portion.

FIG. 11 shows an exemplary concave component of a gauge concave component assembly being used to displaced a concave component in situ to determine a spacing for the implant concave component.

FIG. 12 shows an exemplary implant concave component, having the determined spacing, being inserted and attached to the concave component stem.

FIG. 13 shows a front view of an exemplary joint replacement in situ gauge system for a knee joint having two displacement devices.

FIG. 14 shows a side view of an exemplary joint replacement in situ gauge system for a knee joint.

FIG. 15 shows a perspective view of a joint replacement in situ gauge system having two separate convex components and a gauge concave assembly having two separate concave portions and two corresponding displacement devices.

FIG. 16 shows a perspective view of a portion of a gauge convex assembly having two separate displacement devices and two separate concave portions for receiving the two convex components (not shown).

FIG. 17 shows a front view of a joint replacement in situ gauge system configured to interface with two separate convex components and comprising a gauge convex assembly having two separate displacement devices; wherein the pressure on the gauge is low.

FIG. 18 shows a front view of the joint replacement in situ gauge system shown in FIG. 17 with the pressure increased and the displacement scale showing a higher displacement than the displacement scale; thereby indicating a non-uniform displacement requirement for the implant concave component.

FIG. 19 shows a front view of an implant convex component and concave component having a non-uniformly displaced concave component.

Corresponding reference characters indicate corresponding parts throughout the several views of the figures. The figures represent an illustration of some of the embodiments of the present invention and are not to be construed as limiting the scope of the invention in any manner. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

### DETAILED DESCRIPTION OF THE ILLUSTRATED EMBODIMENTS

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and are illustrated in the accompanying figures. The embodiments described are only for purposes of illustrating the present invention and should not be interpreted as limiting the scope of the invention. Other embodiments of the invention, and certain modifications, combinations and improvements of the described embodiments, will occur to those skilled in the art and all such alternate embodiments, combinations, modifications, improvements are within the scope of the present invention.

### Definitions

A convex portion of a convex component is the curved convex surface of the component and may be a radiused surface including a spherical surface; common with ball type joints.

A concave portion of a concave component is the curved concave surface of the component and may be radiused surface such as a spherically shaped recess common with socket type joints.

In situ is defined as in the natural or original position or place. The gauge component of the joint replacement system is situated in situ with respect to the joint replacement to provide an accurate measurement of the spacing requirements under a given load.

A permanent implant concave or convex component as used herein is a component that is implanted and may be implanted into a portion of the gauge concave or convex assembly after determination of the proper size for the permanent implant component.

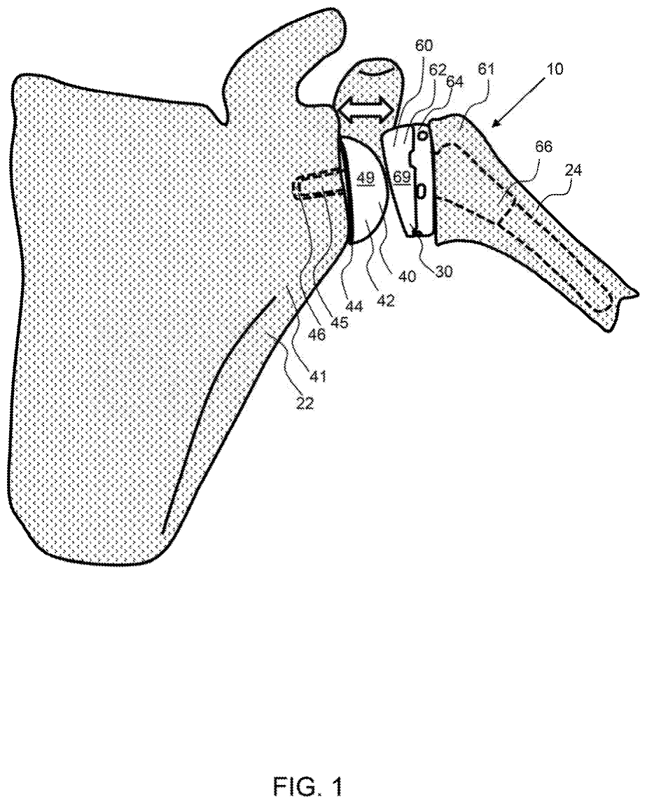

As shown in FIG. 1, an exemplary joint replacement in situ gauge system 10 is configured for a shoulder joint and in particular a reverse shoulder joint replacement. This exemplary joint replacement system comprises a convex component 40 secured to the convex receiver bone 41, or scapula 22, and a concave component secured to the concave receiver bone 61, or humerus 24. The convex component has a convex portion 42 that interfaces with a concave portion 62 of the concave component to produce a ball/socket joint or interface. The convex component is coupled to the stem 46 by a stem insert 45 coupled to the plate portion 42. The concave component is coupled to the stem 66 by a plate portion 64. The convex component assembly 49 and/or the concave component assembly 69 may be a gauge component for determining the proper spacing for an implant convex or concave component.

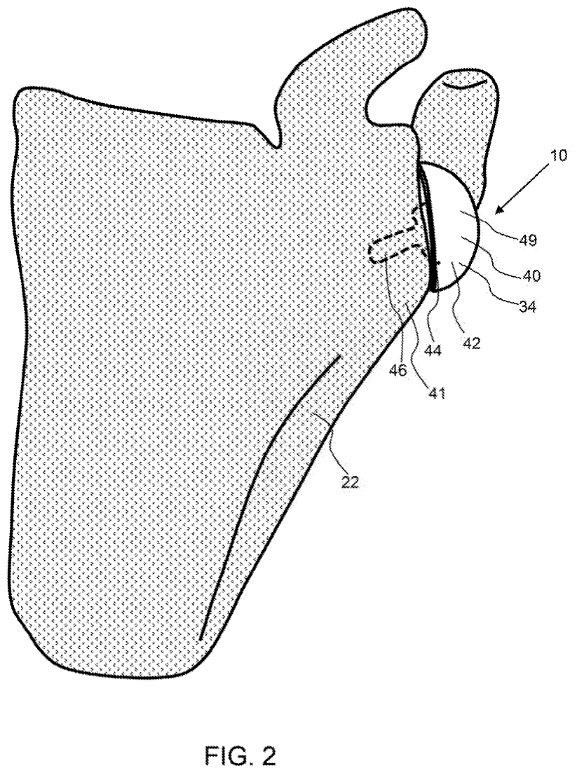

FIG. 2 shows an exemplary convex component assembly 49 of the joint replacement system secured into the scapula 22 by a stem 46. The convex component assembly comprises a convex component having a convex portion and a plate portion and stem. The convex component may have a stem insert that extends into a stem secured within the bone when the convex component is a gauge component. In an exemplary embodiment, a bone component is fixed to a stem and that is secured into the convex receiver bone and the concave component is a gauge component.

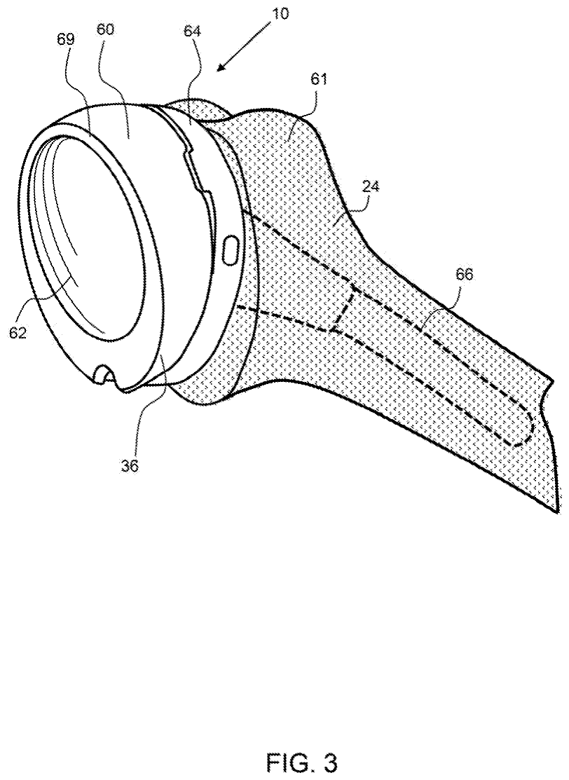

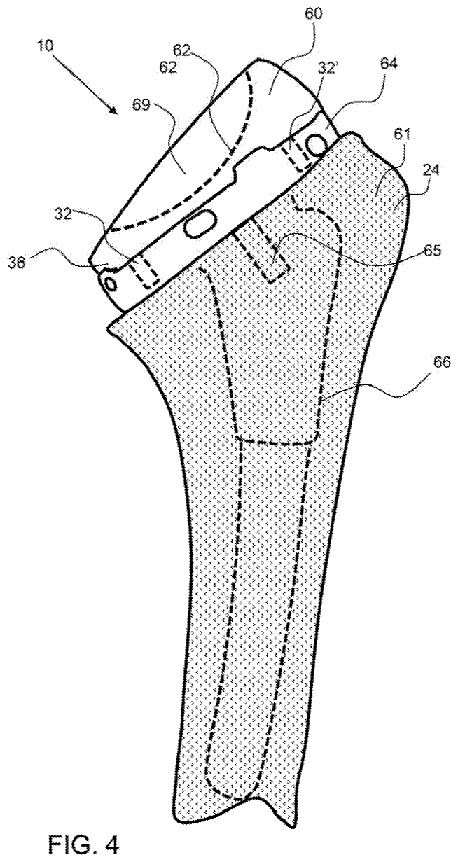

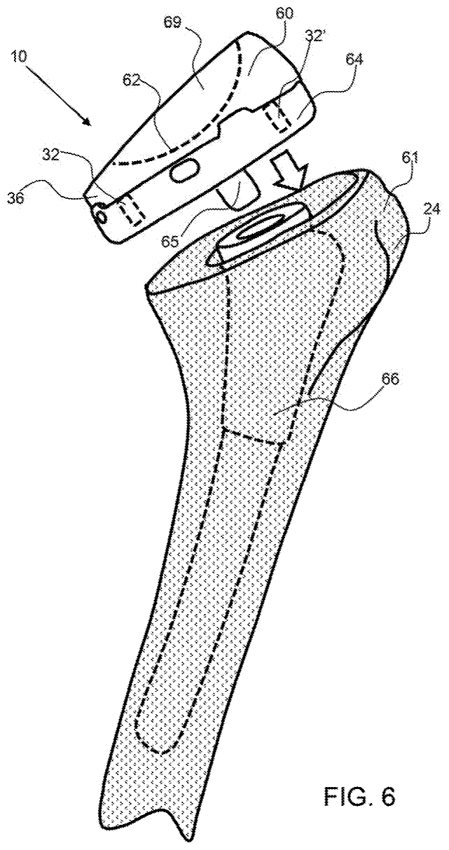

As shown in FIGS. 3 and 4, an exemplary gauge concave assembly 36 of the joint replacement in situ gauge system 10 is secured into the concave receiver bone 61 by a stem insert 65 that extends into the stem 66. The stem extends down into the bone and has an aperture to receive a stem insert, shown in FIG. 6. An exemplary gauge concave assembly comprises a concave component 60 having a concave portion 62 to move about a convex portion of a convex component, a plate portion 64 and a stem insert 65 for insertion into the stem. The stem may be secured and fixed into the concave receiver bone and the gauge concave component may be inserted into the stem for measurement purposes. A plurality of guide posts 32, 32′ extend between the concave component 60 and the plate portion 64 to guide the concave component uniformly out and away from the plate portion and to prevent pitching of the concave component as it is displaced using a displacement device.

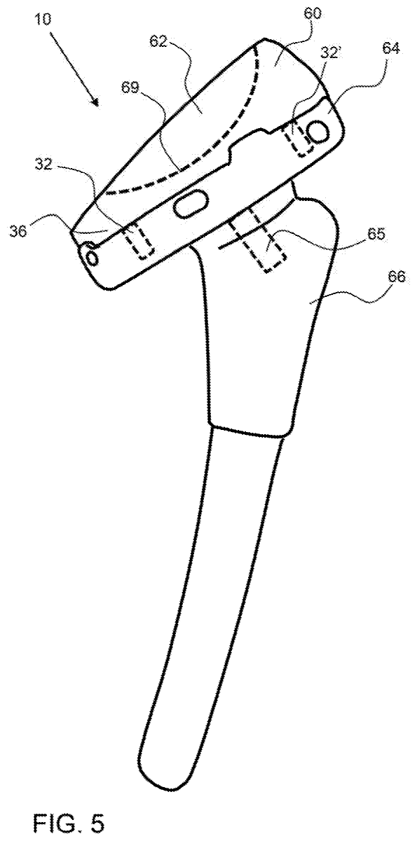

As shown in FIG. 5, an exemplary gauge concave assembly 36 comprises a concave component 60 secured to a plate portion 64 that is detachably attachable to the stem 66.

As shown in FIG. 6, an exemplary gauge concave assembly 36 is being inserted into a stem 66 by the stem insert 65. The stem is secured in a concave receiver bone 61. This detachable attachment enables measurement of spacing and/or forces in situ and subsequent detachment for implantation of an implant concave component have a determined geometry or spacing.

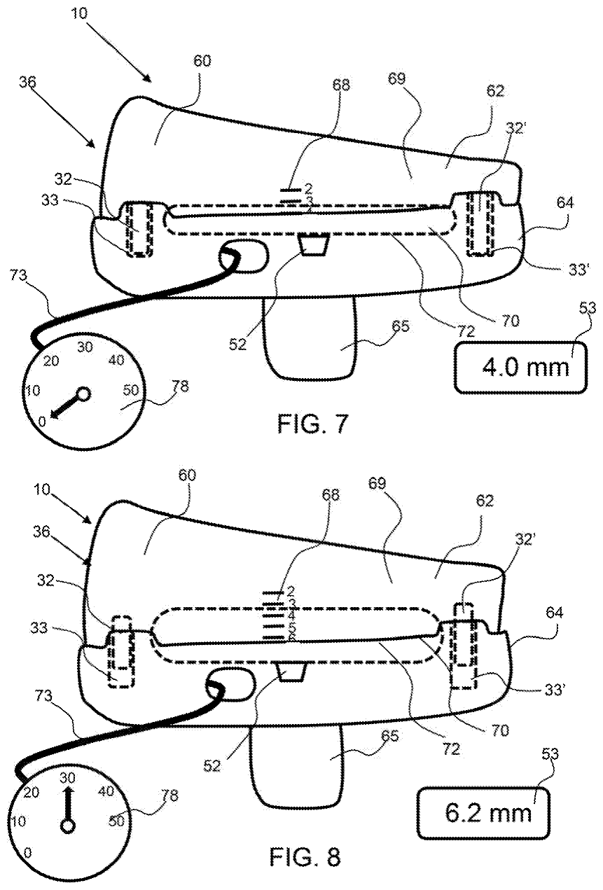

As shown in FIGS. 7 and 8, an exemplary gauge concave assembly 36 has a displacement device 70 to displace or move the concave component 60 with respect to the plate portion 64. As shown in FIG. 8, the displacement device 70, a bladder 72, is extending the concave component away from the plate portion 64. The bladder receives fluid through the fluid line 73 to inflate the bladder and displace the concave component 60. A displacement gauge comprises a displacement scale 68 on the concave component that provides a measurement of spacing that can be used to select an implant concave component. As shown in FIG. 7, the displacement scale is reading 4.0 along the top rim of the plate component and FIG. 8 shows the concave component has been displaced to a reading of 6.2 mm. The gauge concave assembly comprises a displacement scale 68 and a displacement sensor 52 that provides a displacement reading to a receiver 53, such as an electronic device having a read-out of the displacement. A surgeon may use this measurement to determine a proper sized implant concave component. A force gauge 78 may be monitored and a concave component displacement measurement may be taken at a certain force or pressure. The pressure is increased to achieve a desired distraction force on the joint and indicates an effective fit in situ of the concave component with respect to the convex component. A plurality of guide posts 32, 32′ extend between the concave component 60 and the plate portion 64 to guide the concave component uniformly out and away from the plate portion and to prevent pitching of the concave component as it is displaced using a displacement device 70. The guide post(s) may extend from the concave component and into guide apertures 33,33′ in the concave plate portion 64 or vice versa.

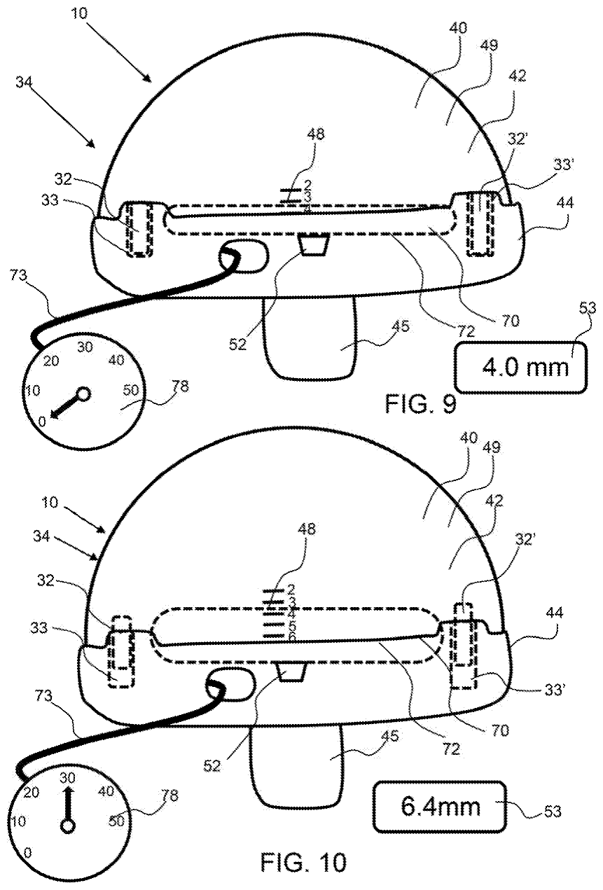

As shown in FIGS. 9 and 10, an exemplary gauge convex assembly 34 has a displacement device 70 to displace or move the convex component 40 with respect to the plate portion 44. As shown in FIG. 10, the displacement device 70, a bladder 72, is extending the convex component away from the plate portion 44. The bladder receives fluid through the fluid line 73 to inflate the bladder and displace the convex component 40. A scale on the convex component provides a measurement of spacing that can be used to select an implant convex component. As shown in FIG. 9, the scale is reading 4.0 mm along the top rim of the plate component and FIG. 10 shows the concave component has been displaced to a reading of about 6.4. The gauge concave assembly comprises a displacement scale 48 and a displacement sensor 52 that provides a displacement reading to a receiver 53, such as an electronic device having a read-out of the displacement. A surgeon may use this measurement to determine a proper sized implant convex component. A gauge 78 may be monitored and a convex component displacement measurement may be taken at a certain pressure. The pressure is increased to achieve a desired distraction force on the joint and indicates an effective fit in situ of the convex component with respect to the concave component. A plurality of guide posts 32, 32′ extend between the convex component 40 and the plate portion 44 to guide the convex component uniformly out and away from the plate portion and to prevent pitching of the convex component as it is displaced using a displacement device 70. The guide post(s) may extend from the convex component and into guide apertures 33,33′ in the convex plate portion 64 or vice versa. The gauge concave assembly comprises a stem insert 45 coupled to the plate portion 44 for securing the gauge concave assembly in a stem.

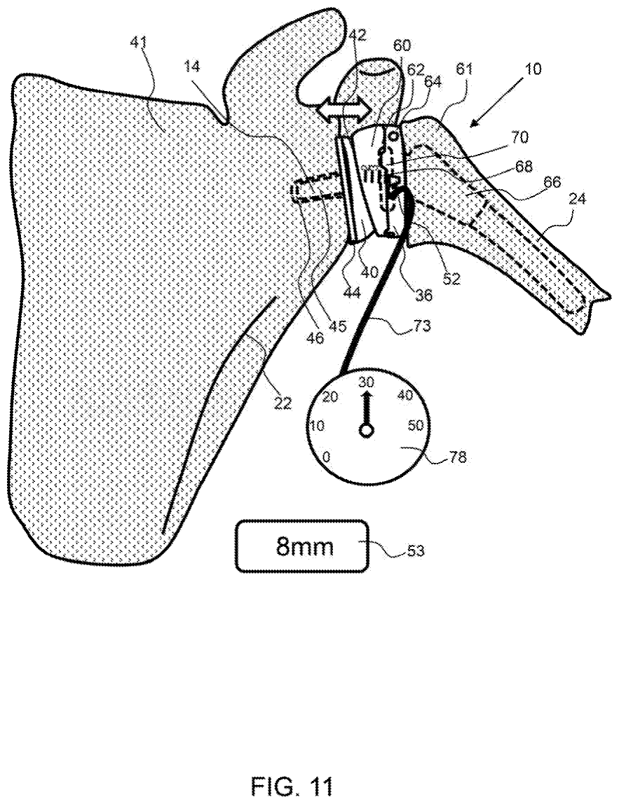

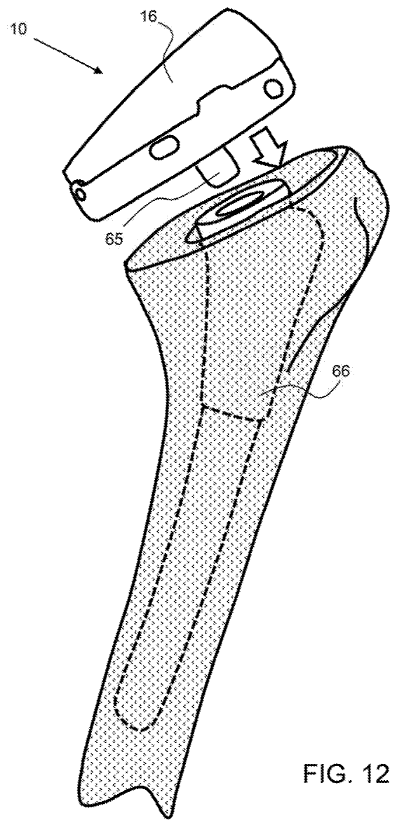

As shown in FIG. 11, an exemplary concave component 60 of a gauge concave component assembly 36 is being displaced in situ to determine a spacing for the implant concave component. The concave component assembly has substantially the same geometric features as the implant concave component, thereby making the reading concave component assembly a natural fit that mimics the actual implant concave component. This in situ gauge joint replacement assembly and method enables quicker determination of proper fits without excessive manipulation of the joint. After a displacement measurement is made, the implant concave component 16 may be implanted and secured to the stem 66 by the implant stem insert 65, as shown in FIG. 12. Again, the implant concave component and implant concave assembly have substantially the same dimensions to enable removal of at least a portion of the gauge concave assembly, such as the gauge concave assembly components.

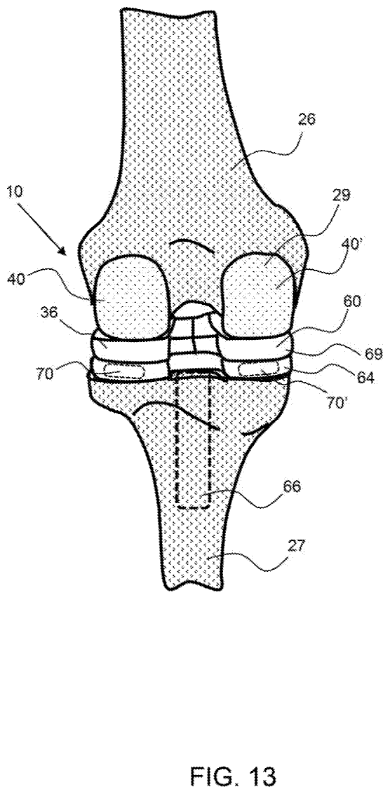

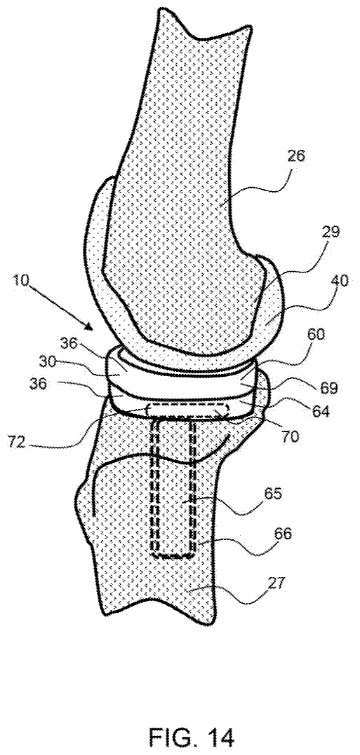

As shown in FIGS. 13 and 14, an exemplary joint replacement in situ gauge system 10 for a knee joint 29 has two displacement devices 70, 70′. The femur 26 is configured with two convex components 40, 40′ and the concave component 60 has two concave portions to receive the two convex components. The two displacement devices are configured in the plate portion 64 of the gauge concave assembly 36 which is secured to the tibia 27 by a stem 66. This gauge component enables measurement of separate displacement distance for each of the two convex components. The concave component may have a non-uniform displacement or thickness between the two convex components.

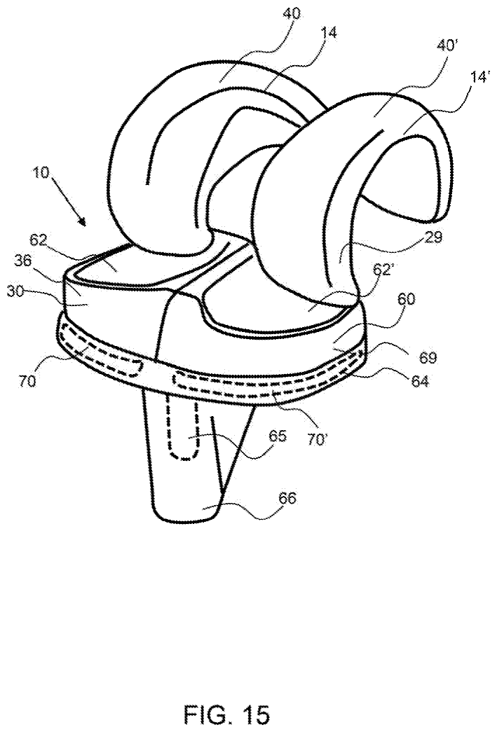

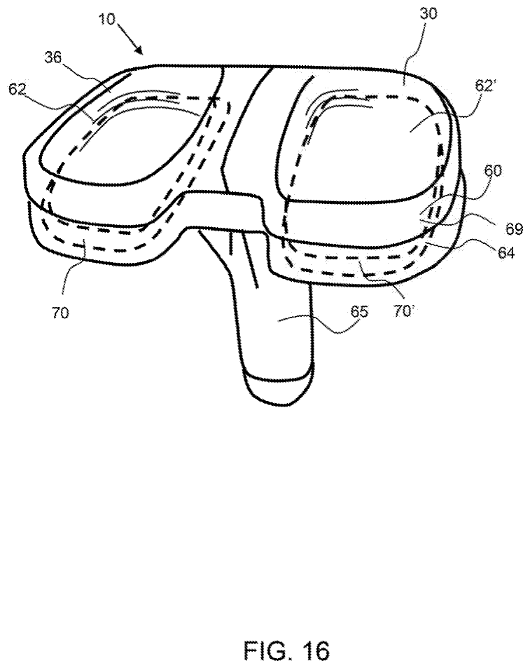

As shown in FIGS. 15 and 16, an exemplary joint replacement in situ gauge system 10 for a knee joint 29 has two displacement devices 70, 70′. The implant convex components 14, 14′ may be secured to the femur and are configured to rest and rotate within the corresponding concave portions 62, 62′ of the concave component 60. The gauge concave assembly 36 has two separate displacement devices 70, 70′, such as bladders, for individual determination of the displacement of the two concave portions or in some cases the two concave components. The stem insert 65 extends down from the plate portion 64 into the stem. The stem is coupled with the bone, such as being inserted into an aperture in the bone.

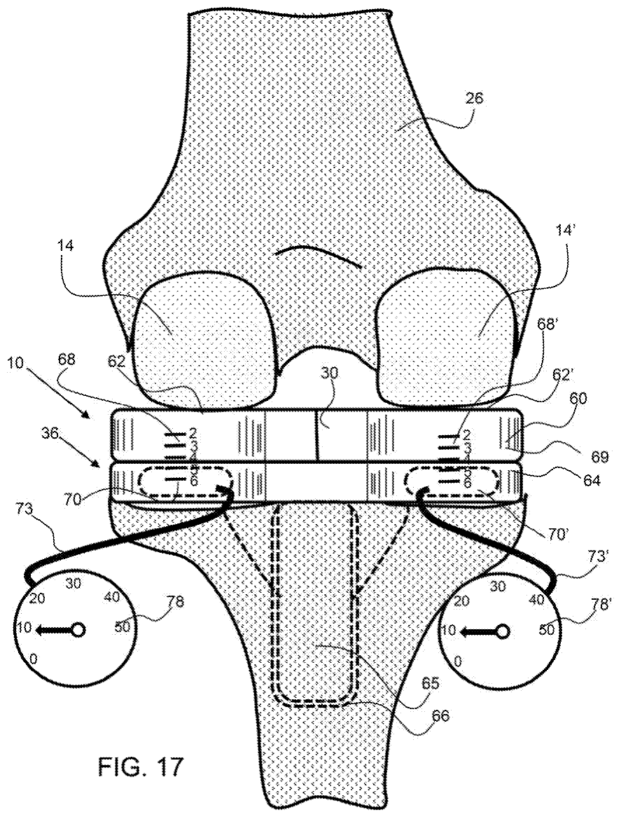

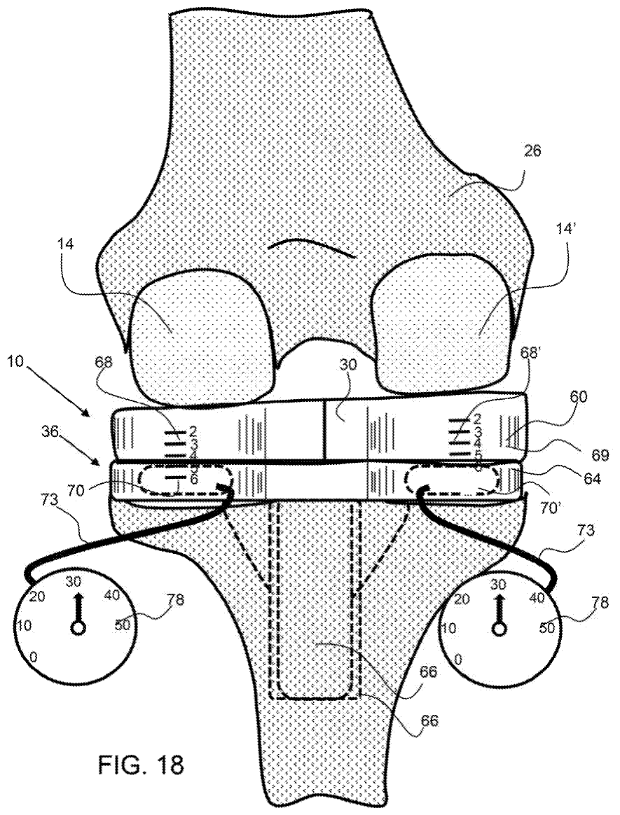

As shown in FIG. 17, a joint replacement in situ gauge system 10 has a gauge concave assembly 36 having two separate displacement devices 70, 70′. The pressure is low and the displacement, as measured by the gauges 78, 78′ is uniform across the concave component 60. The concave portions 62, 62′ are engaged with the implant convex components 14, 14′. However, as shown in FIG. 18, with the gauge pressure increased to a pressure suitable for gauge displacement measurement, scale 68′ is showing a larger displacement than scale 68. This may indicate that a non-uniform implant concave component, or a concave component having two different displacements from the plate component 64, is required. This non-uniform implant concave component may be required for proper medial and lateral knee alignment and pressure distribution.

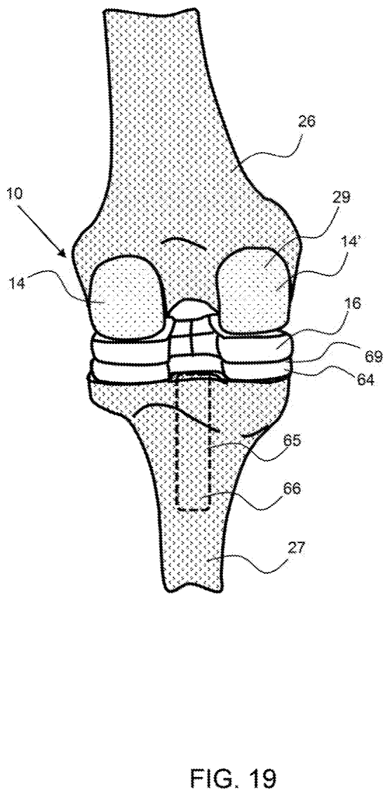

As shown in FIG. 19, the gauge concave assembly 36 is removed and an implant concave component 16 is configured in the knee joint 29.

It will be apparent to those skilled in the art that various modifications, combinations and variations can be made in the present invention without departing from the scope of the invention. Specific embodiments, features and elements described herein may be modified, and/or combined in any suitable manner. Thus, it is intended that the present invention cover the modifications, combinations and variations of this invention provided they come within the scope of the appended claims and their equivalents.

What is claimed is:

1. A joint replacement in situ gauge system comprising:
a) a convex component assembly comprising a convex component having a convex portion;
b) a concave component assembly comprising a concave component having a concave portion;
c) a displacement device configured in one of the convex component assembly or the concave component;
wherein the displacement device displaces the convex portion or the concave portion to determine a proper sized implant.

2. A joint replacement in situ gauge system for a joint comprising:
a) a convex component assembly comprising a convex component having a convex portion; wherein the convex component is coupled to a convex receiver bone;
b) a gauge concave component assembly coupled to a concave receiver bone and comprising:
i) a plate portion detachably attached to the concave receiver bone;
ii) a concave component coupled to the plate portion and having a concave portion;
iii) a displacement device located between the concave component and the plate portion and configured to displace the concave component away from the plate portion in situ;
wherein the convex portion of the convex component is aligned with the concave portion of the concave component;
wherein the displacement device displaces the concave component to determine a proper sized implant concave component.

3. The joint replacement in situ gauge system of claim 2, wherein the gauge concave component assembly further comprises a force gauge to measure a force exerted on the concave component.

4. The joint replacement in situ gauge system of claim 2, wherein the gauge concave component assembly further comprises a displacement gauge to measure a displacement of the concave portion from the plate portion.

5. The joint replacement in situ gauge system of claim 4, wherein the displacement gauge is a physical gauge on the gauge concave component assembly.

6. The joint replacement in situ gauge system of claim 4, wherein the displacement gauge comprises a displacement sensor.

7. (canceled)

8. (canceled)

9. The joint replacement in situ gauge system of claim 2, wherein the displacement device is a bladder that receives a fluid from a pump to inflate the bladder.

10. The joint replacement in situ gauge system of claim 2, wherein the gauge concave component assembly further comprises a guide post extending between the concave component and the plate portion to guide the concave component as it is displaced.

11. The joint replacement in situ gauge system of claim 2, comprising two convex portions and wherein the gauge concave component assembly comprises two concave portions and two displacement devices, wherein the two concave portions can be displaced different amounts from the plate portion.

12. The joint replacement in situ gauge system of claim 11, wherein the joint is a knee joint.

13. A joint replacement in situ gauge system for a joint comprising:
a) a concave component assembly comprising a concave component having a concave portion;
wherein the concave component is coupled to a concave receiver bone;
b) a gauge convex component assembly coupled to a convex receiver bone and comprising:
i) a plate portion detachably attached to the convex receiver bone;
ii) a convex component coupled to the plate portion and having a convex portion;
iii) a displacement device located between the convex component and the plate portion and configured to displace the convex component away from the plate portion in situ;
wherein the convex portion of the convex component is aligned with the concave portion of the concave component;
wherein the displacement device displaces the convex component to determine a proper sized implant convex component.

14. The joint replacement in situ gauge system of claim 13, wherein the gauge convex component assembly further comprises a force gauge to measure a force exerted on the concave component.

15. The joint replacement in situ gauge system of claim 13, wherein the gauge convex component assembly further comprises a displacement gauge to measure a displacement of the convex portion from the plate portion.

16. The joint replacement in situ gauge system of claim 15, wherein the displacement gauge is a physical gauge on the gauge convex component assembly.

17. The joint replacement in situ gauge system of claim 15, wherein the displacement gauge comprises a displacement sensor.

18. (canceled)

19. (canceled)

20. The joint replacement in situ gauge system of claim 13, wherein the displacement device is a bladder that receives a fluid from a pump to inflate the bladder.

21. The joint replacement in situ gauge system of claim 13, wherein the gauge convex component assembly further comprises a guide post extending between the convex component and the plate portion to guide the convex component as it is displaced.

22. A joint replacement method comprising:
a) providing a joint replacement in situ gauge system for a joint comprising:
i) a convex component assembly comprising:
a convex component having a convex portion;
wherein the convex component is coupled to a convex receiver bone;
ii) a gauge concave component assembly coupled to a concave receiver bone and comprising:
a plate portion detachable attached to the concave receiver bone;
a concave component coupled to the plate portion and having a concave portion;
a displacement device located between the concave component and the plate portion and configured to displace the concave component away from the plate portion in situ;
b) aligning the convex portion of the convex component with the concave portion of the gauge concave component;
c) displacing the gauge concave component with the displacement device;
d) measuring a displacement of the concave component;
e) removing the gauge concave component;
f) selecting an implant concave component using the displacement measured; and
g) securing the implant concave component to the concave receiver bone.

23. The joint replacement method of claim 22, wherein the gauge concave component assembly comprises a displacement scale and wherein measuring said displacement of the concave component includes reading the displacement scale.

24. (canceled)

25. The joint replacement method of claim 22, wherein the gauge concave component assembly comprises a displacement sensor that outputs a displacement reading to a receiver and wherein measuring said displacement of the concave component includes reading the displacement reading on the receiver.

26. (canceled)

27. The joint replacement method of claim 22, wherein the gauge concave assembly further comprises a force gauge to measure a force exerted on the concave component as it is displaced by the displacement device.

28. (canceled)

29. (canceled)

30. The joint replacement method of claim 2229, wherein the displacement device is a bladder that receives a fluid from a pump to inflate the bladder.

31. The joint replacement method of claim 30, wherein the gauge concave assembly further comprises a force gauge to measure a force exerted on the concave component as it is displaced by the displacement device and wherein the force is a pressure of said fluid in the bladder.

32. A joint replacement method comprising:
a) providing a joint replacement in situ gauge system for a joint comprising:
i) a concave component assembly comprising:
a concave component having a concave portion;
wherein the concave component is coupled to a concave receiver bone;
ii) a gauge convex component assembly coupled to a convex receiver bone and comprising:
a plate portion detachable attached to the convex receiver bone;
a convex component coupled to the plate portion and having a convex portion;
a displacement device located between the convex component and the plate portion and configured to displace the convex component away from the plate portion in situ;
b) aligning the convex portion of the convex component with the concave portion of the concave component
c) displacing the convex component with the displacement device;
d) measuring a displacement of the convex component;
e) removing the convex component;
f) selecting an implant convex component using the displacement measured; and
g) securing the implant convex component to the convex receiver bone.

33. The joint replacement method of claim 32, wherein the gauge convex component assembly comprises a displacement scale and wherein measuring said displacement of the convex component includes reading the displacement scale.

34. (canceled)

35. The joint replacement method of claim 32, wherein the gauge convex component assembly comprises a displacement sensor that outputs a displacement reading to a receiver and wherein measuring said displacement of the convex component includes reading the displacement reading on the receiver.

36. (canceled)

37. The joint replacement method of claim 32, wherein the gauge convex assembly further comprises a force gauge to measure a force exerted on the convex component as it is displaced by the displacement device.

38. (canceled)

39. (canceled)

40. The joint replacement method of claim 32, wherein the displacement device is a bladder that receives a fluid from a pump to inflate the bladder.

41. The joint replacement method of claim 40, wherein the gauge convex assembly further comprises a force gauge to measure a force exerted on the convex component as it is displaced by the displacement device and wherein the force is a pressure of said fluid in the bladder.
